(19) United States
(12) Patent Application Publication
(10) Pub. No.: US 2021/0369103 A1
(43) Pub. Date: Dec. 2, 2021

(54) System, Method, and Apparatus for Controlling Environment Surrounding Eye

(71) Applicant: Aurora Tears Technology, Inc., Santa Clara, CA (US)

(72) Inventor: Ping Cerina Zhang, Sunnyvale, CA (US)

(21) Appl. No.: 17/191,603

(22) Filed: Mar. 3, 2021

Related U.S. Application Data
(60) Provisional application No. 63/033,031, filed on Jun. 1, 2020.

(51) Int. Cl. A61B 3/10; A61B 3/16; A61B 3/113; A61B 3/00; G02C 7/10; G02C 7/02

(52) U.S. Cl. CPC A61B 3/101; A61B 3/16; A61B 3/113; A61B 3/0025; A61B 2562/0247; G02C 7/02; A61B 2562/029; A61B 2562/0271; G02C 7/102

(57) ABSTRACT

An apparatus comprises a support structure. The apparatus also comprises a plurality of sensors positioned on the support structure. The apparatus also comprises a camera positioned on the support structure. The camera has a field of view that includes an eye of a user. The apparatus also includes one or more processors and memory. The memory stores instructions that, when executed by the one or more processors, cause the processors to detect ambient condition data using the plurality of sensors. The processors also capture imaging data that includes the eye using the camera. The processors determine a first predefined state of the eye based on the detected ambient condition data and the captured imaging data. The processors further dispense a fluid proximate to the eye in accordance with the first predefined state of the eye.

### RELATED APPLICATIONS

This application claims priority to U.S. Provisional Patent Application No. 63/033,031, filed Jun. 1, 2020, titled “System, Method, and Apparatus for Controlling Environment Surrounding Eye,” which is incorporated by reference herein in its entirety.

This application is related to U.S. patent application Ser. No. 16/464,631, filed May 28, 2019, titled “Systems and Methods for Generating and Applying Biomimicry Tear Films,” which is incorporated by reference herein in its entirety.

### TECHNICAL FIELD

The disclosed implementations relate generally to systems, methods, and devices for monitoring a person's eye and an environment around the eye.

### BACKGROUND

Adequate moisture is integral to the overall health of eyes and vision. Blinking is an important body function that provides moisture to the eyes through release of a lubricating tear film. Dry eye syndrome is a condition that occurs when a person's tears are not able to provide adequate lubrication to their eyes

### SUMMARY

Blinking maintains the healthy functioning of the eyes by providing the eyes with moisture. Typically, most people blink their eyes every ten seconds or so. However, the rate of blinking reduces with increased screen time. When people interact with their device screens (e.g., computers, tablets, smart phones, etc.) or watch television, they tend not to blink as much. Prolonged use of the devices can lead to dryness in the eyes, fatigue of the eyes, stinging in the eyes, sensitivity to light, and other irritations. Furthermore, in some circumstances, certain ambient conditions can also cause or aggravate the dry eye syndrome. For example, air-conditioned environments can have lower humidity levels compared to outdoor environments, thus worsening the condition of the eyes.

Because devices with display screens (e.g., computers, laptops, tablets, mobile phones, television etc.) have become indispensable in one's everyday life, users will continue to utilize the devices despite experiencing fatigue and/or discomfort in their eyes. Accordingly, there is a need for improved systems, devices, and methods that monitor an external environment surrounding the eyes of a user and the condition of the eyes themselves. There is also a need for improved systems, devices, and methods that proactively mitigate the condition of the eyes in accordance with the monitoring. Ideally, the systems, devices, and methods should allowing a user to continue using their eyes for their tasks (e.g., working, reading, watching television, cooking etc.) while the mitigation is being carried out.

As disclosed herein, an apparatus (e.g., a device, such as an electronic device) is equipped with sensors that actively monitor a surrounding environment of the eye(s) of a user. The apparatus is also equipped with a camera (e.g., an imaging device) that monitors the condition of the eyes themselves. In some embodiments, the apparatus determines a state of the eye(s) in accordance with the data from the camera. In some embodiments, in accordance with the determined state of the eye(s), the apparatus may take one or more actions to mitigate, maintain, or optimize the condition of the eye(s). For example, the apparatus may regulate the humidity of the environment surrounding the eye(s), and/or dispense one or more fluids to the eyes to maintain the amount of moisture in the eye(s), and/or display an alert to the user, either via a display and/or one or more lenses of the apparatus. In some embodiments, the apparatus can also send the alert to a display device of the user, for display on the device. In some embodiments, the apparatus can also mitigate the surrounding environment of the eye(s), for example by controlling one or more operating conditions (e.g., a temperature, a humidity level etc.) external devices in the vicinity of the user which can affect the level of moisture in the eyes.

In accordance with some embodiments of the present disclosure, an apparatus comprises a support structure. The apparatus also comprises a plurality of sensors positioned on the support structure. The apparatus further comprises a camera positioned on the support structure. The camera has a field of view that includes an eye of a user of the apparatus. The apparatus further comprises one or more processors and memory. The memory stores instructions that, when executed by the one or more processors, cause the processors to detect ambient condition data using the plurality of sensors. The processors also capture imaging data that includes the eye using the camera. The processors determine a first predefined state of the eye based on the detected ambient condition data and the captured imaging data. The processors also dispense a fluid proximate to the eye of the user in accordance with the first predefined state of the eye.

In some embodiments, the apparatus further comprises one or more liquid reservoirs positioned on the support structure. Dispensing the fluid proximate to the eye of the user comprises dispensing the fluid from the one or more liquid reservoirs.

In some embodiments, the apparatus further comprises one or more agitators positioned in proximity to the one or more liquid reservoirs. Dispensing the fluid proximate to the eye of the user further comprises agitating the fluid in the one or more liquid reservoirs prior to the dispensing the fluid.

In some embodiments, the one or more agitators include one or more of: a radio frequency resonator, a magnetic mixer, and an ultrasonic vibrator.

In some embodiments, the one or more liquid reservoirs include one or more micro-heaters. The memory further includes instructions that, when executed by the one or more processors, cause the processors to adjust a temperature of the fluid in the one or more liquid reservoirs using the one or more micro-heaters.

In some embodiments, the memory further includes instructions that, when executed by the one or more processors, cause the processors to dispense the fluid from the one or more liquid reservoirs by evaporating the fluid using the one or more micro heaters.

In some embodiments, the one or more liquid reservoirs comprise a plurality of liquid reservoirs. Each of the plurality of liquid reservoirs contains a distinct fluid having a corresponding fluid type. In accordance with the determined first predefined state of the eye, the processors identify one or more fluid types corresponding to the first predefined state. The processors also dispense from the plurality of liquid reservoirs fluids one or more fluids corresponding to the identified fluid types.

In some embodiments, the ambient condition data includes two or more of: a light level, an air pressure, humidity, an air flow, and temperature. The plurality of sensors include two or more of: a light sensor for measuring the light level; an ambient pressure sensor for measuring the air pressure; a humidity sensor for measuring the humidity; an airflow sensor for measuring the air flow; and a temperature sensor for measuring the temperature.

In some embodiments, the apparatus further comprises a tonometer for measuring a pressure of the eye.

In some embodiments, the tonometer comprises a first component for deflecting a cornea of the eye. The tonometer also comprises a second component for measuring the deflection.

In some embodiments, the apparatus further comprises a refractor for measuring an intraocular pressure of the eye.

In some embodiments, the memory further includes instructions that, when executed by the one or more processors, cause the processors to determine one or more parameters from the imaging data. In some embodiments, the one or more parameters include: a blinking rate of the eye; a color of the eye; secretion from the eye; swelling of the eye; a size of a pupil of the eye; and cloudiness of the eye.

In some embodiments, the support structure includes an engagement mechanism for engaging the support structure in a vicinity of the eye.

In some embodiments, the apparatus further comprises one or more lenses mounted on the support structure.

In some embodiments, the one or more lenses include photochromic lenses. The memory further includes instructions that, when executed by the one or more processors, cause the processors to vary a shade of the photochromic lenses in accordance with the determined first predefined state of the eye.

In some embodiments, the one or more lenses are configured to display one or more indications to the user, including: light signals, text, and/or images.

In some embodiments, the apparatus further comprises communication circuitry for communicatively connecting the apparatus with an electronic device. The memory further includes instructions that, when executed by the one or more processors, cause the processors to transmit the ambient condition data and/or the images to the electronic device for display on the electronic device.

In some embodiments, the memory further includes instructions that, when executed by the one or more processors, cause the processors to store the ambient condition data and the images on the apparatus.

In some embodiments, the apparatus further comprises a battery and a charging port.

In accordance with another aspect of the present disclosure, a method is performed at an apparatus. The apparatus includes a support structure, a plurality of sensors positioned on the support structure, and a camera positioned on the support structure. The camera has a field of view that includes an eye of a user. The apparatus also includes comprises one or more processors and memory. The memory stores one or more programs configured for execution by the one or more processors. The method comprises detecting ambient condition data using the plurality of sensors. The method also comprises capturing imaging data that includes the eye using the camera. The method also comprises determining a first predefined state of the eye based on the detected ambient condition data and the captured imaging data. The method further comprises dispensing a fluid proximate to the eye in accordance with the first predefined state of the eye.

In some embodiments, a non-transitory computer-readable storage medium stores one or more programs configured for execution by an apparatus (e.g., an electronic device) having one or more processors and memory. The one or more programs include instructions for performing any of the methods described herein.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an exemplary perspective view of an apparatus for controlling an environment surrounding an eye of a user in accordance with some embodiments.

FIG. 2 illustrates a side view of the apparatus in accordance with some embodiments.

FIG. 3 illustrates a front view of the apparatus in accordance with some embodiments.

FIG. 4 illustrates a back view of the apparatus in accordance with some embodiments.

FIG. 5 illustrates an exemplary view of a graphical user interface in accordance with some embodiments.

FIG. 6 illustrates a block diagram of an apparatus in accordance with some embodiments.

FIG. 7 illustrates a flowchart of a method performed at an apparatus in accordance with some embodiments.

Reference will now be made to implementations, examples of which are illustrated in the accompanying drawings. In the following description, numerous specific details are set forth in order to provide a thorough understanding of the present invention. However, it will be apparent to one of ordinary skill in the art that the present invention may be practiced without requiring these specific details.

### DESCRIPTION OF IMPLEMENTATIONS

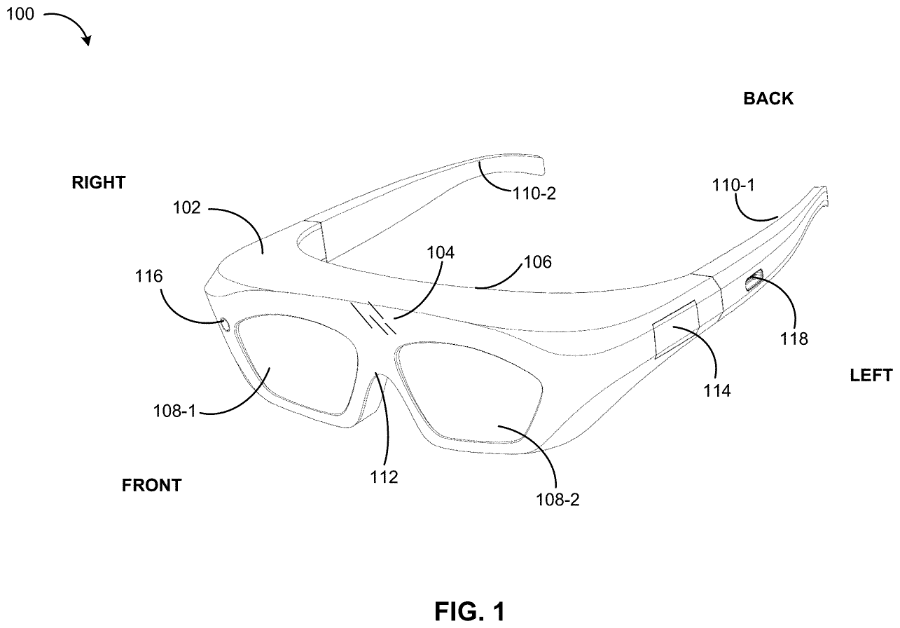

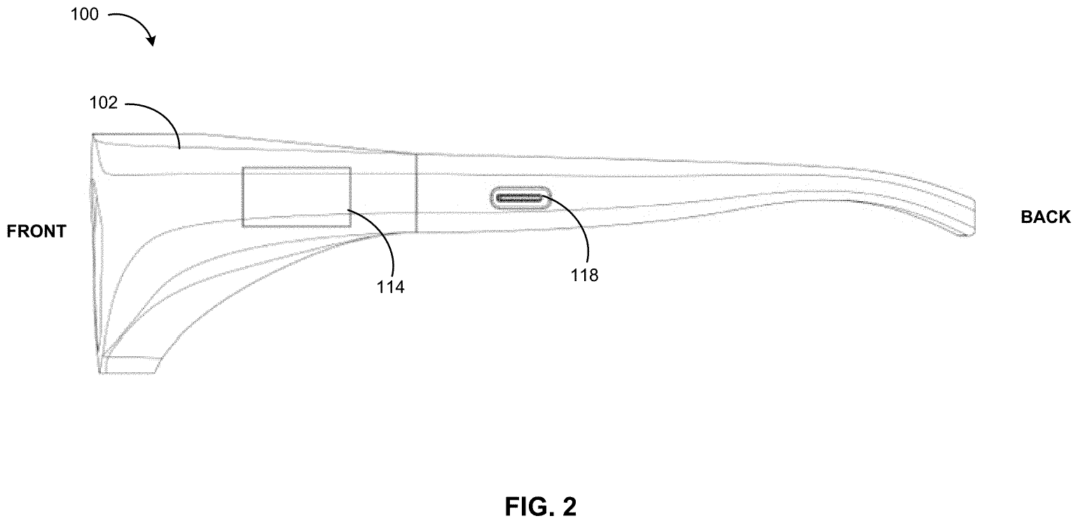

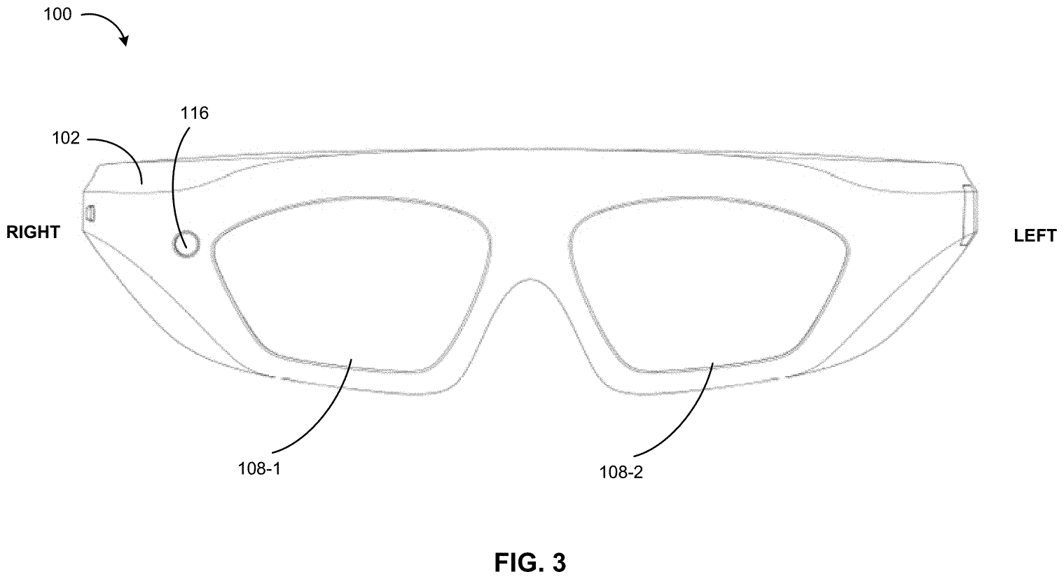

FIGS. 1, 2, and 3 illustrate, respectively, exemplary perspective, side, and front views of an apparatus 100 for controlling an environment surrounding an eye of a user in accordance with some embodiments. In some embodiments, the apparatus 100 is mounted directly on a user's head. In some embodiments, the apparatus 100 comprises a wearable apparatus (e.g., wearable accessory) that is worn by a user, such as a pair of goggles, spectacles, or eye glasses, etc.

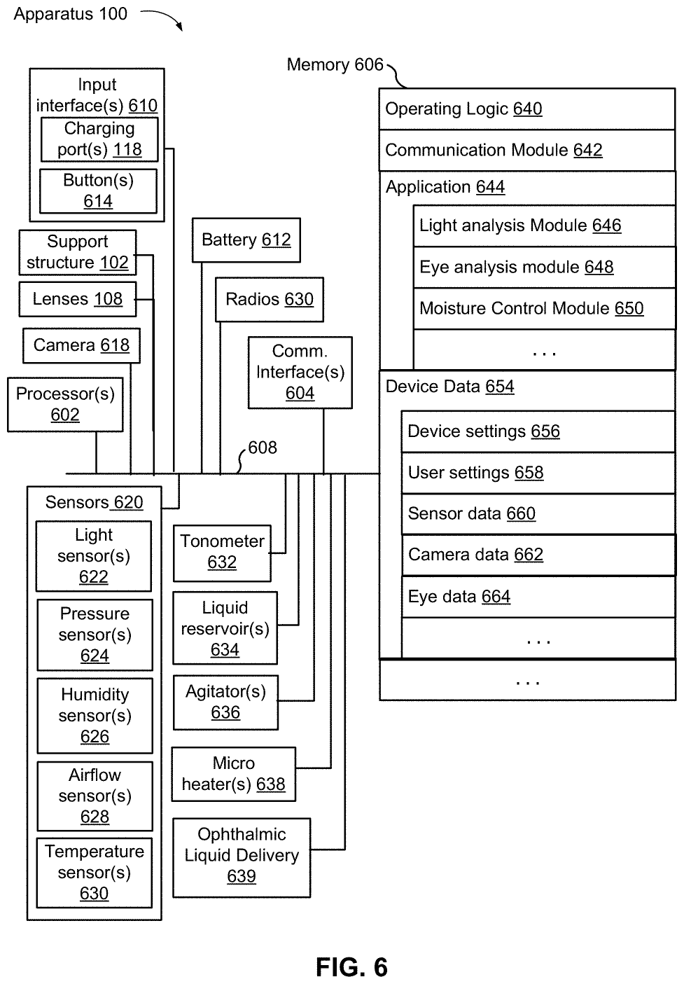

In some embodiments, the apparatus 100 comprises a support structure 102. In some embodiments, and as illustrated in FIG. 1, the support structure 102 comprises a frame that includes an exterior surface 104 (e.g., an exterior-facing side) that faces away from the user's eye and toward an external environment surrounding the user's head. In some embodiments, the exterior surface 104 includes sensor openings that exposes one or more sensors (e.g., sensors 620, FIG. 6) toward a surrounding of the user, to ambient conditions in proximity to the user, such as airflow, light, air pressure, humidity, temperature, etc.

FIG. 1 also shows that the support structure 102 comprises an interior surface 106 (e.g., an interior-facing side) that faces a direction toward the user's eye(s), in accordance with some embodiments. In some embodiments, the interior surface 106 includes sensor openings that exposes one or more sensors (e.g., sensors 620, FIG. 6) toward the user's eye(s), to facilitate monitoring of a condition of the user's eye(s).

In some embodiments, the support structure 102 includes one or more engagement mechanisms for engaging one or more portions of a user's head to the apparatus 100. For example, in some embodiments the engagement mechanisms include temples 110 (e.g., a left temple 110-1 and a right temple 110-2), which are “arm” pieces coupled to the support structure 102 that extend over and/or behind the ears of the user, to hold the support structure 102 in place. In some embodiments, the engagement mechanisms include a bridge 112 that arches over the nose of the user, between lenses 108. In some embodiments, the bridge 112 supports a majority of the weight of the apparatus 100.

In some embodiments, the support structure 102 also includes one or more lenses 108, such as a left lens 108-1 and a right lens 108-2, which are mounted on the support structure 102. In some embodiments, the lenses 108 comprise non-prescription lenses that do not contain a prescription correction. In some embodiments, the lenses 108 comprise prescription lenses that are customized for the eyes of the user.

In some embodiments, the lenses 108 comprise photochromic lenses that vary in shade in accordance with the surrounding environment. For example, in some embodiments, the lenses 108 darken in the presence of sunlight and lighten when there is reduced or no light in accordance with some embodiments. In some embodiments, the lenses 108 comprise photochromic lenses that vary in shade in accordance with a determined state of the eye. In another example, the lenses 108 can turn dark in accordance with a determination (e.g., via the processor(s) 602 analyzing images acquired by a camera 618, FIG. 6) that the pupil of an eye appears dilated in the presence of sunlight. For example, after surgery, the eye may stay dilated for up to a day before returning to normal. Accordingly, in circumstances like these, the lenses 108 may adjust a shade to protect the eye(s) from overexposure to light.

In some embodiments, the lenses 108 includes basic display functions, and are capable of displaying text and/or light. For example, in some instances, in accordance with a determination that the user has been using their computer for an extended period of time, the apparatus 100 can display via the lenses 108 an alert (e.g., in the form of text and/or flashing light) to the user to take a break, and/or reduce the brightness level of the computer screen. In some embodiments, the apparatus 100 also includes one or more processors and one or more communication interfaces (e.g., processor(s) 602 and communication interface(s) 604, FIG. 6) that transmits the alert signals to a connected device, for display on that device.

In some embodiments, the apparatus 100 includes the support structure 102 and not the lenses 108 (e.g., the apparatus comprises a frame without lens). For example, in some circumstances, a user may not require corrective eyeglasses would like a means of introducing some form of relief to the eye. In some embodiments, one or more eye patches may be placed on or over the support structure 102 to protect the eye(s).

In some embodiments, the apparatus 100 includes various components (e.g., devices, units, modules, etc.) that are affixed on (e.g., coupled to, held on) the support structure 102 at certain positions relative to the user's head. The components can measure parameters of an environment surrounding the eye(s), and/or measure parameters of the eye(s) themselves, in accordance with some embodiments.

In some embodiments, the apparatus 100 includes a moisture control unit 114 for monitoring (e.g., measuring) an amount of (e.g., a level of) moisture in an area proximate to the user's eye(s). The moisture control unit 114 can also adjust the amount of moisture in the environment surrounding the user's eye(s) in accordance with the amount of measured moisture, to prevent the eye(s) from drying up.

FIG. 1 shows that the moisture control unit 114 is positioned on the support structure 102, adjacent to one of the temples 110 (e.g., the left temple 110-1) in accordance with some embodiments. Alternatively, the moisture control unit can be positioned on the support structure 102, adjacent to the right temple 110-2, or anywhere on the support structure 102. In some embodiments, the apparatus 100 can include two moisture control units, each positioned on either side (e.g., left and right sides) of the apparatus 100 and in the vicinity of the user's eye. In some embodiments, the moisture control unit 114 is embedded in the support structure 102. In some embodiments, the moisture control unit 114 is external add-on unit (e.g., module) and can be attached on the apparatus 100 to be used as needed.

In some embodiments, the moisture control unit 114 (e.g., the apparatus 100) includes sensors for detecting ambient conditions in a surrounding environment of the user. FIG. 6 illustrates a block diagram of the apparatus 100 in accordance with some embodiments. In some embodiments, and as illustrated in FIG. 6, the apparatus includes sensors 620, such as pressure sensor(s) 624, humidity sensor(s) 626 (e.g., hygrometer, psychometer etc.), airflow sensor(s) 628, and/or temperature sensors(s) 630. The sensors 620 detect environmental conditions and/or parameters that can impact an amount of moisture surrounding the eye(s). For example, the pressure sensor(s) 624 are used to measure air pressure. In some embodiments, a higher air pressure measurement is indicative of lower humidity.

In some embodiments, the sensors 620 include humidity sensor(s) 626 (e.g., hygrometer, psychrometer etc.), which are used to monitor the humidity near the eye (e.g., inside the enclosure of the apparatus 100, or between the eyes and the lenses 108 of the apparatus 100, etc.) and a humidity of the environment (e.g., outside the enclosure of the apparatus 100, in front of the apparatus 100, etc.). In some embodiments, the apparatus 100 (e.g., the moisture control unit 114) includes a plurality of humidity sensors 626 positioned at different locations (e.g., at different locations on the interior side 106 and the exterior side 104) of the apparatus 100, for measuring humidity in the immediate vicinity and the surrounding environment of the user's eye, and to adjust a humidity level when necessary. For example, in one scenario, the moisture control unit 114 can increase the humidity in accordance with a determination that a lower humidity is measured on the exterior side 104 than on the interior side 106, because the difference in humidity indicates that the immediate vicinity of the user's eye will likely become drier over time. In another scenario, the moisture control unit 114 can maintain the humidity (e.g., maintain the amount of moisture that is dispensed) in accordance with a determination that humidity is measured on the exterior side 104 and the interior side 106 are about equal, and/or if a lower humidity is measured on the interior side 106 than on the exterior side 104.

In some embodiments, the sensors 620 also include airflow sensor(s) 628 for measuring air flowing in the vicinity of the apparatus 102. In some circumstances, higher airflow can lead to increased evaporation rates, and therefore lower moisture levels.

The sensors 620 also include temperature sensor(s) 630, in accordance with some embodiments. The temperature sensor(s) 630 are used to monitor the temperature near the eye (e.g., inside the enclosure of the apparatus 100, or between the eyes and the lenses 108 of the apparatus 100, etc.) and a temperature of the environment surrounding the eye (e.g., outside enclosure of the apparatus 100 or in front of the apparatus 100, etc.). In some embodiments, the moisture control unit 114 (e.g., the apparatus 100) includes a plurality of temperature sensors 630 positioned at different locations (e.g., at different locations on the interior side 106 and exterior side 104) of the apparatus 100).

With continued reference to FIGS. 1 and 6, in some embodiments, the moisture control unit 114 includes one or more liquid reservoir(s) 634 (see FIG. 6) that stores fluid(s) that can be used by the eye(s) and/or to control the environment surrounding the eye(s). A fluid includes moisture, air, water, water vapor, artificial tears, prescription medicine, ophthalmic liquid, tear film, distinct layers of the tear film (e.g., lipid layer, aqueous layer, and mucin layer), and/or any substance that flows, etc. For example, in some embodiments, the liquid reservoir(s) 634 can include container(s) with holes for fluid (e.g., liquid) storage and evaporation, in accordance with some embodiments. In some embodiments, the container itself can be made of metal, plastic, or other non-absorptive materials. In some embodiments, the liquid reservoir 634 is located at one position on the support structure 102 and is connected to liquid channels and evaluation sites at other locations of the support structure 102. In some embodiments, the inside of the container includes highly water/liquid absorbing materials such as sponge, polyvinyl alcohol (PVA), etc. which can be used to further control a volume of the fluid and/or an evaporation speed of the fluid. In some embodiments, the liquid reservoir 634 optionally includes multiple chambers containing different types of fluids (e.g., liquids, such as water, artificial tears, prescriptive eye drops, etc.), which can be premixed prior to dispensing. In some embodiments, the respective chambers individually dispense liquids and/or vapors to the surrounding area of the eye(s) without pre-mixing.

In some embodiments, the apparatus 100 includes two or more liquid reservoirs 634, each containing a respective type of liquid. The apparatus 100 can dispense one or more respective liquids, or pre-mix at least two of the liquids prior to dispensing. Various details of a liquid reservoir for dispensing artificial tears or other liquid to the eye are described in U.S. patent application Ser. No. 16/464,631, filed May 28, 2019, titled “Systems and Methods for Generating and Applying Biomimicry Tear Films,” which is hereby incorporated by reference herein in its entirety. Similar mechanisms can be utilized to add moisture to the area near the eye by the apparatus 100, in some embodiments.

In some embodiments, the moisture control unit 114 also includes one or more agitators (e.g., agitator(s) 636, FIG. 6) that are positioned in proximity to (e.g., inside of, next to, etc.) the one or more liquid reservoirs 634. The agitator(s) 634 can comprise a device or mechanism that places fluid(s) in the liquid reservoir(s) 634 to motion, such as by agitating, shaking, swirling, stirring, heating, etc. For example, in some embodiments, the agitator(s) 636 may include a radio frequency resonator, which causes the fluid(s) in the liquid reservoir(s) 634 to vibrate e.g., at a certain frequency. In some embodiments, the agitator(s) 636 may include a magnetic mixer, such as a magnetic agitator resonator and/or a magnetic stirrer etc. In some embodiments, the agitator(s) 636 may include an ultrasonic vibrator.

In some embodiments, the moisture control unit 114 also includes one or more micro heaters (e.g., micro heater(s) 638, FIG. 6). In accordance with some embodiments, the micro heater(s) 638 can heat up fluids inside the liquid reservoir(s) 634. For example, in some embodiments, a liquid reservoir may contain water, which can be heated and evaporated by the micro heater(s) 638, thereby increasing an amount of moisture around the inside surface 106 to moisten the user's eye. In some embodiments, the micro heater(s) 638 can be used to adjust a temperature of a corresponding fluid inside the liquid reservoir(s) 634 prior to dispensing the liquid. For example, the temperature of the micro heater(s) 638 can be controlled via an input control (e.g., input device(s) 610, button(s) 614, etc.) of the apparatus 100, or by another external and/or peripheral device that is communicatively connected to the apparatus 100 through the communication interface(s) 604.

In some embodiments, and as illustrated in FIG. 1, the apparatus 100 includes a light detection unit 116. The light detection unit 116 includes light sensor(s) 622 (see FIG. 6) that detect (e.g., sense, monitor, etc.) and record (e.g., measure) a light level in proximity to the apparatus 100. The light sensors(s) 622 can include photoresistor-, photodetector- and/or light emitting diode (LED)-based sensors in accordance with some embodiments. In some embodiments, the light sensor(s) 622 may be positioned on the support structure 102, in the same plane (e.g., on the same surface) as the lenses 108, to monitor incident light entering the apparatus 100 through the lenses 108. In some embodiments, the light sensor(s) 622 may be positioned on a side of the support structure 102 to monitor the surrounding light (e.g., light that is not directly in the user's eyes), and/or inside the apparatus 100 (e.g., on the inside surface 106) to monitor the incident light inside the apparatus 100.

As further illustrated in FIG. 1, in some embodiments, the apparatus 100 includes a charging port 118. For example, the charging port 118 can comprise a micro USB port, a magnetic connector, or a wireless charger. In some embodiments, the charging port 118 is part of a power unit (e.g., power unit 648, FIG. 6) of the apparatus 100. The power unit 648 may also comprise a battery 612 (e.g., a rechargeable battery), and the charging port 118 connects the apparatus 100 to a power source (e.g., via a cable) to recharge the battery 612.

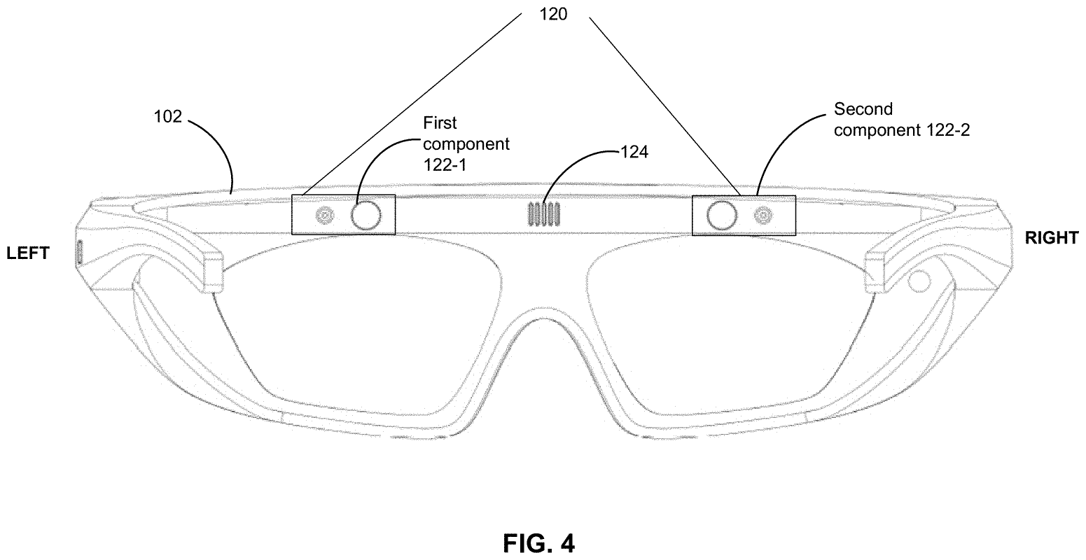

FIG. 4 illustrates a back view (e.g., looking from the back toward the front direction) of the apparatus 100 in accordance with some embodiments.

In some embodiments, and as illustrated in FIG. 4, the apparatus 100 includes an eye monitoring unit 120 (e.g., an eye monitoring and/or checking unit) for monitoring the eye(s) themselves. In the example of FIG. 4, the eye monitoring unit 120 includes a first eye monitoring component 122-1 and a second eye monitoring component 122-2, for monitoring the left eye and the right eye, respectively. In accordance with some embodiments, the eye monitoring unit 120 includes a camera 618 (see FIG. 6). In some embodiments, the camera 618 has an image sensor with a field of view that includes both eyes. In some embodiments, the camera 618 includes two image sensors, such that one of the image sensors has a field of view that includes one eye and the other image sensor has a field of view that includes the other eye. In some embodiments, the eye monitoring unit 120 includes two cameras, each positioned on a respective eye monitoring component 122. The camera 618 is capable of capturing images and/or video of the eye(s). The camera 618 can be used to monitor the frequency of eye-blinking and the visible changes of the topical surface of the eye and eyelid, like the redness, eye discharge, presence of stye, etc. In some embodiments, when combined with a light source (e.g., an LED light) on the support structure 102 on the frame or with an external light source, the camera 618 (e.g., either by itself or with an additional micro lens) can be used for anterior segments imaging or fundus photography by capturing images that include the eye(s) and saving these images (e.g., camera data 622, FIG. 6) locally on the apparatus 100 or on an external storage device. In some embodiments, the camera data 662 (e.g., images and/or video) can be used for ophthalmic examination and for monitoring relevant diseases, such as glaucoma, cataract, tumors, trauma, diabetics, etc.

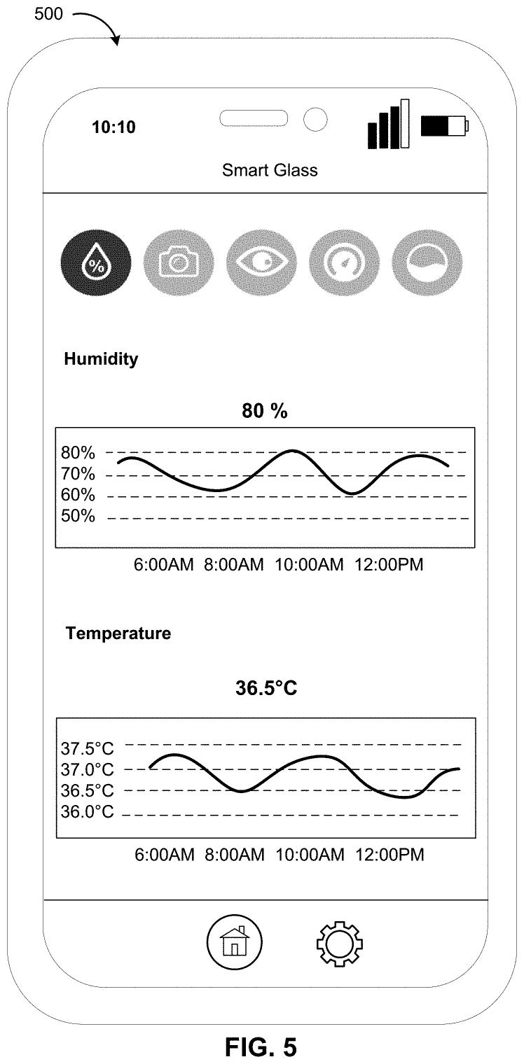

FIG. 4 also shows that the apparatus 100 includes a communication unit 124 (e.g., communication interface(s) 604, communication module 642, etc., FIG. 6) in accordance with some embodiments. The communication unit 124 sends signals and/or information such as data collected by the sensors 620 and/or the camera 618 to the Cloud and/or other connected devices (e.g., electronic device 500, FIG. 5) for storage and/or display. In one example, in accordance with a determination that the humidity level in the surrounding environment is too low, the apparatus 100 may, through the communication unit 124, send a signal to a connected humidifier, to activate the humidifier, in accordance with some embodiments. In another example, in accordance with a determination that a temperature in the surrounding environment is higher than usual, which may lead to a higher evaporation rate, the apparatus 100 may, through the communication unit 124, send a signal to a connected thermostat, to decrease a temperature set point of the environment. In some embodiments, the communication unit 124 also receives signals and and/or information from other connected devices, such as electronic device 500 in FIG. 5.

FIG. 5 illustrates an exemplary user interface on an electronic device 500 in accordance with some embodiments. The electronic device can be a tablet, a mobile phone, a laptop, a display assistant device, or any electronic device that includes a display screen. The user interface is part of an application program executes on the electronic device 500, for displaying data collected by the sensors 620 and/or the camera 618. In some embodiments, the user interface displays parameters such as a humidity level (e.g., relative humidity percentage), temperature, airflow, air pressure, light levels and/or light intensity, a rate of eye-blinking, presence of stye, gunk etc. around the eye, a color of the eyes, a size of the pupils etc. In some embodiments, the user interface also displays alerts to the user to take a break, adjust a screen brightness etc. In some embodiments, the application program also includes options for the user to input one or more parameters, such as a desired humidity level around an interior surface of the support structure 102 and/or a shade of the photochromic lenses 108. The user inputs will then be transmitted from the electronic device to the apparatus 100 for execution by the apparatus 100.

FIG. 6 illustrates a block diagram of an apparatus 100 for in accordance with some embodiments.

The apparatus 100 includes a support structure 102 and lenses 108 as described with respect to FIG. 1, in accordance with some embodiments.

The apparatus 100 also includes one or more processor(s) 602, one or more communication interface(s) 604 (e.g., network interface(s)), memory 606, and one or more communication buses 608 for interconnecting these components (sometimes called a chipset).

In some embodiments, the apparatus 100 includes input interface(s) 610 that facilitates user input. For example, in some embodiments, the input interface(s) 610 include(s) charging port(s) 118 (e.g., FIG. 1) and button(s) 614.

In some embodiments, the apparatus 100 includes a camera 618. The camera 618 has a field of view that includes the eye(s) of the user of the apparatus. In some embodiments, the camera 618 is configured to capture images in color. In some embodiments, the camera 618 is configured to capture images in black and white.

In some embodiments, the apparatus 100 also optionally includes a micro lens for anterior segments imaging or fundus photography, as discussed with respect to FIG. 4.

In some embodiments, the apparatus 100 includes a battery 612. The apparatus 100 also includes sensors 620, such as light sensor(s) 622, pressure sensor(s) 624, humidity sensor(s) 626, airflow sensor(s) 628, and/or temperature sensor(s) 630, as discussed with respect to FIGS. 1 to 4. In some embodiments, the apparatus 100 also includes liquid reservoir(s) 634, agitator(s) 636, and/or micro heater(s) 638, which are described with respect to FIG. 1.

In some embodiments, the apparatus 100 includes radios 630. The radios 630 enable one or more communication networks, and allow the apparatus 100 to communicate with other devices, such as the electronic device 500 in FIG. 5. In some implementations, the radios 630 are capable of data communications using any of a variety of custom or standard wireless protocols (e.g., IEEE 802.15.4, Wi-Fi, ZigBee, 6LoWPAN, Thread, Z-Wave, Bluetooth Smart, ISA100.5A, WirelessHART, MiWi, Ultrawide Band (UWB), software defined radio (SDR) etc.) custom or standard wired protocols (e.g., Ethernet, HomePlug, etc.), and/or any other suitable communication protocol, including communication protocols not yet developed as of the filing date of this document.

In accordance with some embodiments, the apparatus 100 also includes a tonometer 632 (e.g., a tonometer unit). The tonometer 632 can be either a component that is part of the apparatus 100 (e.g., embedded in the support structure 102) or as external unit that is added onto (e.g., attached to) the apparatus 100 to be used as needed. The tonometer 632 measures an intraocular pressure of the eye (e.g., fluid pressure inside the eye). Eye pressure is a critical parameter for establishing the presence of chronical eye disease, such as glaucoma, cataract, etc., which can lead to vision loss if untreated. In some embodiments, the tonometer 632 is used for detecting the presence of chronical eye disease and/or for monitoring the progression of the disease. The tonometer 632 can comprise non-contact air puff tonometry, applanation tonometry, or probe contact tonometry technology etc. In some embodiments, the tonometer 632 can include a micro-pressure sensor that is coupled with an optical system.

In some embodiments, the tonometer 632 optionally includes functions of alignment. The tonometer 632 includes components that cause deflection of the eye cornea, and/or components to measure the deflection, in accordance with some embodiments.

For example, in some embodiments, the tonometer 632 includes a micro probe with precision position control, which contacts a cornea surface and applies a pressure to the eye, thereby causing cornea deflection. In some embodiments, the tonometer 632 includes a micro pump that generates an air puff, which is directed through a micro nozzle to apply pressure to the eye, thereby causing cornea deflection. The cornea surface deflection can be measured by an optical system, which includes an IR light source and a light detector. The light received by the detector varies along with the cornea surface deflection. Here, an IR sensor and a photodetector can be used for serving these functions. In air puff non-contact tonometry, a micro reflected air pressure sensor can be used to measure the reflect air to measure the cornea deflection, in accordance with some embodiments.

In some embodiments, the tonometer 632 also includes a CCD camera, which is used for aligning the center of the eye with the probe or air tube. In some embodiments, a CMOS camera sensor is used to capture the image of the eye surface and to align the center of the eye with the micro probe or air nozzle mentioned above, in accordance with some embodiments.

In some embodiments, the apparatus 100 also includes an ophthalmic liquid delivery module (OLDM) 639. The OLDM 639 can be embedded in the support structure 102 or as external add-on module to be used as needed. The OLDM 639 can be made by an inkjet printing apparatus, or a fluid spray ejector device. Here, instead of conventional inkjet printing technology whereby ink is printed on paper, the inkjet printing apparatus or fluid spray ejector device is used to dispense ophthalmic liquid onto the eye, in accordance with some embodiments.

The OLDM 639 can include an actuation motor and an ophthalmic liquid module (OLM) for delivering ophthalmic liquid, in accordance with some embodiments. The OLM can include a motor, such as a micro piezo motor or piston motor, for providing actuation to pump (e.g., push) the liquid inside OLM to dispense/spray towards the eye through a micro nozzle or micro muzzle array. The angle and dispense range of OLDM 639 can be designed based on the distance and angle between the micro nozzle to the eye. In some embodiments, the motor also facilitates removal and disposal of the liquid after each use. In some embodiments, the OLDM is either embedded in or mounted on the support structure 102; accordingly the OLDM is positioned close to the eye (e.g., within a range of few millimeters to a few centimeters).

The memory 606 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. The memory 606, optionally, includes one or more storage devices remotely located from one or more processor(s) 602. The memory 606, or alternatively the non-volatile memory within the memory 906, includes a non-transitory computer-readable storage medium. In some implementations, the memory 606, or the non-transitory computer-readable storage medium of the memory 606, stores the following programs, modules, and data structures, or a subset or superset thereof:
operating logic 640 including procedures for handling various basic system services and for performing hardware dependent tasks;
a communication module 642 (e.g., a radio communication module) for connecting to and communicating with other network devices (e.g., a local network, such as a router that provides Internet connectivity, networked storage devices, network routing devices, server systems electronic device 500, and/or other connected devices etc.) coupled to one or more communication networks via the communication interface(s) 604 (e.g., wired or wireless);
application 644 for detecting an environment surrounding the eye(s) as well as a condition of the eye(s) themselves, and for controlling one or more components of the apparatus 100 and/or other connected devices in accordance with a determined state of the eye. In some embodiments, the application 644 includes:
   a light analysis module 646 for analyzing signals (e.g., ambient light levels, light intensities etc.) from the light sensor(s) 622. In some embodiments, the light analysis module 646 adjusts a shade of the lenses 108 in accordance with the detected light levels;
   an eye analysis module 648 for analyzing a condition of the eye(s) based on images and/or video captured by the camera 618, and/or based on ambient condition data collected by the sensors 620. In some embodiments, the eye analysis module 648 determines a state of the eye based on images, video, and/or ambient condition data. For example, in some embodiments, the state of the eye can include a dry eye state (e.g., based on dryness of the eye), a pink eye state (e.g., based on a color of eye), a discharge eye state (e.g., discharge from the eye), and a swollen eye state (e.g., based on swelling of the eye); and
   a moisture control module 650 for adjusting moisture surrounding the eye(s) based on the condition of the eye(s), the ambient condition data, and/or the state of the eye. For example, in some embodiments, the moisture control module 650 dispenses one or more fluids from the liquid reservoir(s) 634 in accordance with the determined condition of the eye. In some embodiments, the moisture control module 650 sends a signal to evaporate water from one of the liquid reservoir(s) 634 to increase humidity around the eye(s); and
device data 938 for the apparatus 100, including but not limited to:
   device settings 656 for the apparatus 100, such as default options and preferred user settings; and
   user settings 658, such as a preferred level of humidity, and/or a preferred shade for the lenses 108 (e.g., photochromic lenses);
   sensor data 660 that are acquired (e.g., measured) from the sensors 620;
   camera data 662 that are acquired from the camera 618; and
   eye data 664. For example, in some embodiments, the eye data 664 include mapping (e.g., correlations) between a color of the eye and fluid(s) (e.g., from the liquid reservoir(s) 634) to be applied to the eye, dryness in the eye and fluid(s) (e.g., from the liquid reservoir(s) 634) to be applied to the eye, a type/color of discharge from the eye and fluid(s) (e.g., from the liquid reservoir(s) 634) to be applied to the eye. In some embodiments, the eye data also includes data on predefined states of the eye. For example, the predefined states can include a dry eye state (e.g., based on dryness of the eye), a pink eye state (e.g., based on a color of eye), a discharge eye state (e.g., discharge from the eye), and a swollen eye state (e.g., based on swelling of the eye) in accordance with some embodiments.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various implementations. In some implementations, the memory 606 stores a subset of the modules and data structures identified above. Furthermore, the memory 606 may store additional modules or data structures not described above. In some embodiments, a subset of the programs, modules, and/or data stored in the memory 606 are stored on and/or executed by a server system, and/or by an external device (e.g., electronic device 500).

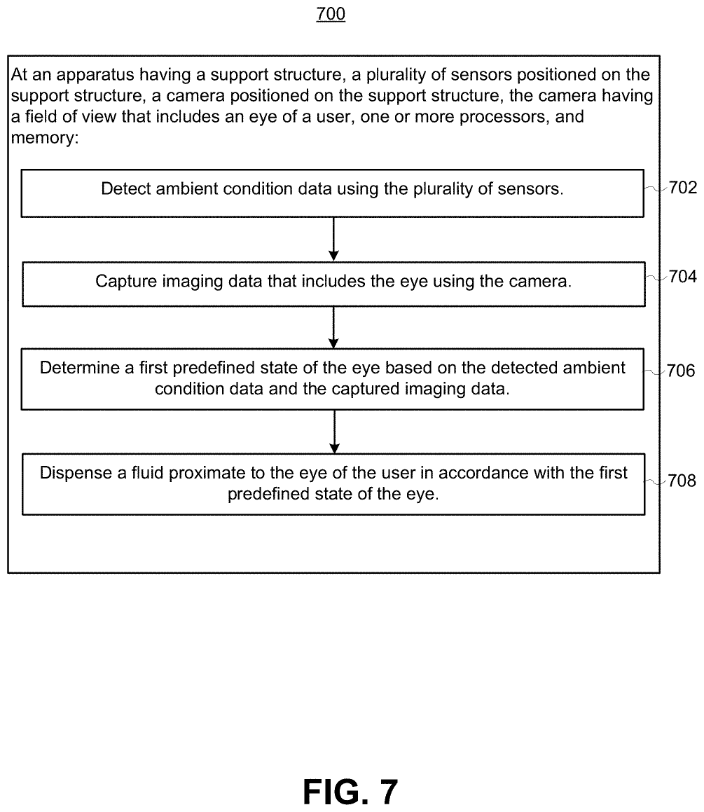

FIG. 7 illustrates a flowchart of a method 700 performed at an apparatus (e.g., apparatus 100, FIGS. 1 to 5), in accordance with some embodiments.

The apparatus 100 comprises a support structure (e.g., support structure 102, FIGS. 1-4). The apparatus 100 also comprises a plurality of sensors (e.g., sensors 620, FIG. 6) positioned on the support structure. The apparatus 100 further comprises a camera (e.g., camera 618, FIG. 6) positioned on (e.g., mounted on, embedded within, integrated into) the support structure. The camera has a field of view that includes an eye of a user of the apparatus 100. The apparatus 100 further comprises one or more processors (e.g., processor(s) 602, FIG. 6) and memory (e.g., memory 606, FIG. 6). The memory stores instructions that are executed by the one or more processors.

The apparatus 100 detects (702) (e.g., senses and measures) (e.g., in real time) ambient condition data using the plurality of sensors, in accordance with some embodiments. For example, in some embodiments, the ambient condition data includes a light level, air pressure, humidity, air flow, and/or temperature.

In some embodiments, the ambient condition data includes two or more of: a light level, an air pressure, humidity, an air flow (e.g., air velocity), and temperature. The plurality of sensors include two or more of: a light sensor (e.g., an ambient light sensor, light sensor(s) 622, FIG. 6) for measuring the light level; an ambient pressure sensor (e.g., pressure sensor(s) 624, FIG. 6) for measuring the air pressure; a humidity sensor (e.g., humidity sensor(s) 626, FIG. 6) for measuring the humidity; an airflow sensor (e.g., airflow sensor(s) 628, FIG. 6) for measuring the air flow; and a temperature sensor (e.g., temperature sensor(s) 630, FIG. 6) for measuring the temperature.

In some embodiments, the apparatus 100 captures (704) imaging data that includes the eye using the camera (e.g., camera 618). For example, the imaging data includes images and video data.

In some embodiments, the apparatus 100 determines (706) a first predefined state of the eye based on the detected ambient condition data and the captured imaging data. For example, the first state of the eye is a first predefined state of a plurality of predefined states. In some embodiments, the predefined states include: a dry eye state (e.g., based on dryness of the eye), a pink eye state (e.g., based on a color of eye), a discharge eye state (e.g., discharge from the eye), and a swollen eye state (e.g., based on swelling of the eye).

In some embodiments, the apparatus 100 dispenses (708) (e.g., automatically, without user invention etc.) a fluid proximate to the eye of the user in accordance with the first predefined state of the eye. For example, fluid includes moisture, air, water, water vapor, artificial tears, prescription medicine, ophthalmic liquid, tear film, distinct layers of the tear film (e.g., lipid layer, aqueous layer, and mucin layer etc.), and/or any substance that flows.

In some embodiments, the apparatus 100 further comprises one or more liquid reservoirs (e.g., liquid reservoir(s) 634, FIG. 6) positioned on the support structure. Dispensing the fluid proximate to the eye of the user comprises dispensing the fluid from the one or more liquid reservoirs.

In some embodiments, the apparatus 100 further comprises one or more agitators (e.g., agitator(s) 636, FIG. 6) positioned in proximity to the one or more liquid reservoirs. Dispensing the fluid proximate to the eye of the user further comprises agitating the fluid in the one or more liquid reservoirs prior to the dispensing the fluid.

In some embodiments, the one or more agitators include one or more of: a radio frequency resonator, a magnetic mixer (e.g., a magnetic agitator resonator, magnetic stirrer etc.), and an ultrasonic vibrator.

In some embodiments, the one or more liquid reservoirs include one or more micro-heaters (e.g., micro heater(s) 638, FIG. 6). The memory further includes instructions that, when executed by the one or more processors, cause the processors to adjust (e.g., change, modify, etc.) a temperature of the fluid in the one or more liquid reservoirs using the one or more micro-heaters.

In some embodiments, the memory further includes instructions that, when executed by the one or more processors, cause the processors to dispense the fluid from the one or more liquid reservoirs by evaporating the fluid using the one or more micro heaters.

In some embodiments, the one or more liquid reservoirs comprise a plurality of liquid reservoirs. Each of the plurality of liquid reservoirs contains a distinct fluid having a corresponding fluid type (e.g., artificial tears, water, air, lipid etc.). In some embodiments, in accordance with the determined first predefined state of the eye, the processors identify one or more fluid types corresponding to the first predefined state. The processors also dispense from the plurality of liquid reservoirs fluids one or more fluids corresponding to the identified fluid types.

In some embodiments, the memory 606 also stores mapping relationships between each of the predefined states of the user's eye and the fluid to use for each of the states (e.g., as eye data 664). For example, dryness of the eye may be mapped to artificial tears, redness of the eye can be correlated to prescription medicine, ophthalmic liquid, etc.

In some embodiments, the apparatus 100 further comprises a tonometer (e.g., tonometer 632, FIG. 6) for measuring a pressure (e.g., an intraocular pressure) of the eye. In some embodiments, the tonometer is embedded in the support structure 102. In some embodiments, the tonometer is an external add-on module that is attached to the support structure 102. In some embodiments, the tonometer comprises non-contact air puff tonometry, applanation tonometry or probe contact tonometry technology. In some embodiments, the tonometer includes a micro pressure sensor coupled with an optical system.

In some embodiments, the tonometer comprises a first component for deflecting a cornea (e.g., a cornea surface) of the eye. The tonometer also comprises a second component for measuring the deflection.

For example, in some embodiments, the tonometer 632 includes a micro probe with precision position control, which contacts a cornea surface and applies a pressure to the eye, thereby causing cornea deflection. In some embodiments, the tonometer 632 includes a micro pump that generates an air puff, which is directed through a micro nozzle to apply pressure to the eye, thereby causing cornea deflection.

In some embodiments, the deflection of a cornea surface can be measured by an optical system that includes an IR light source and a light detector. The light received by the light detector varies along with the cornea surface deflection. In some embodiments, an IR sensor and a photodetector can be used for serving these functions. In air puff non-contact tonometry, a micro reflected air pressure sensor can be used to measure the reflect air to measure the cornea deflection, in accordance with some embodiments. In some embodiments, a CCD camera is used for aligning the center of the eye with the probe or air tube. In some embodiments, a CMOS camera sensor is used to capture the image of the eye surface and used for alignment with the micro probe or air nozzle mentioned above, in accordance with some embodiments.

In some embodiments, the apparatus 100 further comprises a refractor for measuring an intraocular pressure of the eye.

In some embodiments, the memory further includes instructions that, when executed by the one or more processors, cause the processors to determine one or more parameters from the imaging data. In some embodiments, the one or more parameters include: a blinking rate of the eye; a color of the eye (e.g., in particular, redness of the eye); secretion from the eye (e.g., secretion includes tears, discharge of the eye etc.); swelling of the eye; a size of a pupil of the eye (e.g., dilation or contraction of the pupil); and cloudiness of the eye. In some embodiments, the camera 618 is configured to capture images in color. In some embodiments, the camera 618 is configured to capture images in black and white. In this instances, a camera that captures images in black and white, all the parameters except the color of the eye can be captured.

In some embodiments, the support structure 102 includes an engagement mechanism for engaging the support structure in a vicinity of the eye. For example, as illustrated in FIG. 1, the engagement mechanism can include temples 110, which are “arm” pieces coupled to the support structure 102 that extend over and/or behind the ears of the user, to hold the support structure 102 in place. In some embodiments, the engagement mechanism can include a bridge 112 that arches over the nose of the user, between the lenses 108.

In some embodiments, the apparatus 100 further comprises one or more lenses (e.g., lenses 108, FIGS. 1, 3, 4, and 6) mounted on the support structure 102. For example, the one or more lenses can comprise corrective (e.g., prescription) lenses that are customized according to the user's vision conditions, such as myopia, hyperopia, astigmatism and presbyopia etc., in accordance with some embodiments. In some embodiments, the lenses are non-prescription lenses (e.g., the user has normal vision and does not require the use of corrective lenses).

In some embodiments, the one or more lenses include photochromic lenses. The memory further includes instructions that, when executed by the one or more processors, cause the processors to vary (e.g., change, adjust, etc.) a shade of the photochromic lenses in accordance with the determined first predefined state of the eye.

In some embodiments, the one or more lenses are configured to display one or more indications to the user, including: light signals (e.g., blinking, flashing lights etc.), text, and/or images.

In some embodiments, the apparatus 100 further comprises communication circuitry (e.g., communication interface(s) 604, a communication module 642, radios 630 etc., FIG. 6) for communicatively connecting the apparatus with an electronic device (e.g., electronic device 500, FIG. 5). The memory further includes instructions that, when executed by the one or more processors, cause the processors to transmit the ambient condition data and/or the images to the electronic device for display on the electronic device.

In some embodiments, the memory further includes instructions that, when executed by the one or more processors, cause the processors to store the ambient condition data and the images on the apparatus 100 (e.g., as sensor data 660 and/or camera data 662, FIG. 6). In some embodiments, the ambient condition data and the camera data are stored on the electronic device.

In some embodiments, the apparatus further comprises a battery (e.g., battery 612, FIG. 6) and a charging port (e.g., charging port(s) 118, FIGS. 1 and 6). For example, the charging port(s) can comprise a micro USB, a magnetic connector, or wireless charger etc., in accordance with some embodiments.

In accordance with some implementations, a non-transitory computer-readable storage medium (e.g., within the memory 606) stores one or more programs, the one or more programs comprising instructions, which when executed by an apparatus (e.g., apparatus 100), cause the apparatus to perform any of the above methods and/or operations.

Clause 1. An apparatus, comprising: a support structure; a plurality of sensors positioned on the support structure; a camera positioned on the support structure, the camera having a field of view that includes an eye of a user of the apparatus; one or more processors; and memory storing instructions that, when executed by the one or more processors, cause the processors to: (1) detect via the plurality of sensors ambient condition data; (2) capture via the camera imaging data that includes the eye; (3) determine a first predefined state of the eye based on the detected ambient condition data and the captured imaging data; and (4) dispense a fluid proximate to the eye of the user in accordance with the first predefined state of the eye

Clause 2. The apparatus of clause 1, further comprising one or more liquid reservoirs positioned on the support structure; wherein dispensing the fluid proximate to the eye of the user comprises dispensing the fluid from the one or more liquid reservoirs.

Clause 3. The apparatus of clause 2, further comprising one or more agitators positioned in proximity to the one or more liquid reservoirs; wherein dispensing the fluid proximate to the eye of the user further comprises agitating the fluid in the one or more liquid reservoirs prior to the dispensing the fluid

Clause 4. The apparatus of clause 3, wherein the one or more agitators include one or more of: a radio frequency resonator, a magnetic mixer, and an ultrasonic vibrator.

Clause 5. The apparatus of clause 2 or clause 3, wherein the one or more liquid reservoirs include one or more micro-heaters; and the memory further includes instructions that, when executed by the one or more processors, cause the processors to adjust a temperature of the fluid in the one or more liquid reservoirs using the one or more micro-heaters.

Clause 6. The apparatus of clause 5, wherein the memory further includes instructions that, when executed by the one or more processors, cause the processors to: dispense the fluid from the one or more liquid reservoirs by evaporating the fluid using the one or more micro heaters.

Clause 7. The apparatus of clause 2, wherein: the one or more liquid reservoirs comprise a plurality of liquid reservoirs, each of the liquid reservoirs containing a distinct fluid having a corresponding fluid type; and the memory further includes instructions that, when executed by the one or more processors, cause the processors to: (1) in accordance with the determined first predefined state of the eye: (a) identify one or more fluid types corresponding to the first predefined state; and (b) dispense from the plurality of liquid reservoirs fluids one or more fluids corresponding to the identified fluid types.

Clause 8. The apparatus of any of clauses 1-7, wherein: the ambient condition data includes two or more of: a light level, an air pressure, humidity, an air flow, and temperature; and the plurality of sensors include two or more of: a light sensor for measuring the light level; an ambient pressure sensor for measuring the air pressure; a humidity sensor for measuring the humidity; an airflow sensor for measuring the air flow; and a temperature sensor for measuring the temperature.

Clause 9. The apparatus of any of clauses 1-8, further comprising: a tonometer for measuring a pressure of the eye.

Clause 10. The apparatus of any of clause 9, wherein the tonometer comprises: a first component for deflecting a cornea of the eye; and a second component for measuring the deflection.

Clause 11. The apparatus of any of clauses 1-10, further comprising a refractor for measuring an intraocular pressure of the eye.

Clause 12. The apparatus of any of clauses 1-11, wherein the memory further includes instructions that, when executed by the one or more processors, cause the processors to: determine one or more parameters from the imaging data, the one or more parameters including: (1) a blinking rate of the eye; (2) a color of the eye; (3) secretion from the eye; (4) swelling of the eye; (5) a size of a pupil of the eye; and (6) cloudiness of the eye.

Clause 13. The apparatus of any of clauses 1-12, wherein the support structure includes an engagement mechanism for engaging the support structure in a vicinity of the eye.

Clause 14. The apparatus of any of clauses 1-13, further comprising one or more lenses mounted on the support structure.

Clause 15. The apparatus of clause 14, wherein the one or more lenses include photochromic lenses; and the memory further includes instructions that, when executed by the one or more processors, cause the processors to vary a shade of the photochromic lenses in accordance with the determined first predefined state of the eye.

Clause 16. The apparatus of clause 14 or clause 15, wherein the one or more lenses are configured to display one or more indications to the user, including: light signals, text, and/or images.

Clause 17. The apparatus of any of clauses 1-13, further comprising: communication circuitry for communicatively connecting the apparatus with an electronic device; and the memory further includes instructions that, when executed by the one or more processors, cause the processors to transmit the ambient condition data and/or the images to the electronic device for display on the electronic device.

Clause 18. The apparatus of clause 17, wherein the memory further includes instructions that, when executed by the one or more processors, cause the processors to store the ambient condition data and the images on the apparatus.

Clause 19. The apparatus of any of clauses 1-18, further comprising a battery and a charging port.

Clause 20. A method, comprising: at an apparatus having a support structure, a plurality of sensors positioned on the support structure, a camera positioned on the support structure, the camera having a field of view that includes an eye of a user, one or more processors, and memory storing one or more programs configured for execution by the one or more processors, the method comprising: (1) detecting via the plurality of sensors ambient condition data; (2) capturing via the camera imaging data that includes the eye; (3) determining a first predefined state of the eye based on the detected ambient condition data and the captured imaging data; and (4) dispensing a fluid proximate to the eye in accordance with the first predefined state of the eye.

Clause 21. The method of clause 20, wherein the apparatus further comprises one or more liquid reservoirs positioned on the support structure, the method further comprising: dispensing the fluid from the one or more liquid reservoirs.

Clause 22. The method of clause 21, wherein the apparatus further comprises one or more agitators positioned in proximity to the one or more liquid reservoirs, the method further comprising: prior to dispensing the fluid, agitating the fluid in the one or more liquid reservoirs.

Clause 23. The method of clause 21 or clause 22, wherein the one or more liquid reservoirs include one or more micro-heaters, the method further comprising: adjusting a temperature of the fluid in the one or more liquid reservoirs using the one or more micro-heaters.

Clause 24. The method of clause 23, further comprising: dispensing the fluid from the one or more liquid reservoirs by evaporating the fluid using the one or more micro heaters.

Clause 25. The method of any of clauses 21-24, wherein the one or more liquid reservoirs comprise a plurality of liquid reservoirs, each of the liquid reservoirs containing a distinct fluid having a corresponding fluid type, the method further comprising: in accordance with the determined first predefined state of the eye: identifying one or more fluid types corresponding to the first predefined state; and dispensing from the plurality of liquid reservoirs fluids one or more fluids corresponding to the identified fluid types.

Clause 26. The method of any of clauses 20-25, wherein the ambient condition data includes two or more of: a light level, an air pressure, humidity, an air flow and temperature; and the plurality of sensors include two or more of: (1) a light sensor for measuring the light level; (2) an ambient pressure sensor for measuring the air pressure; (3) a humidity sensor for measuring the humidity; (4) an airflow sensor for measuring the air flow; and (5) a temperature sensor for measuring the temperature.

Clause 27. The method of any of clauses 20-26, wherein the apparatus further includes a tonometer for measuring a pressure of the user's eye.

Clause 28. The method of clause 27, wherein the tonometer includes a first component and a second component, the method further comprising: deflecting a cornea of the eye via the first component; and measuring the deflection via the second component

Clause 29. The method of any of clauses 20-28, further comprising determining one or more parameters from the imaging data, the one or more parameters including: a blinking rate of the eye; a color of the eye; secretion from the eye; swelling of the eye; a size of a pupil of the eye; and cloudiness of the eye.

Clause 30. The method of any of clauses 20-29, wherein the support structure includes one or more photochromic lenses, the method further comprising: varying a shade of the photochromic lenses in accordance with the determined first predefined state of the eye.

Clause 31. The method of any of clauses 20-30, wherein the frame includes one or more lenses, the method further comprising: displaying on the one or more lenses one or more indications to the user, including: light signals, text, and/or images.

Clause 32. The method of any of clauses 20-31, wherein the apparatus is communicatively connected to an electronic device, the method further comprising: transmitting the ambient condition data and/or the images to the electronic device for display on the electronic device.

Clause 33. The method of clause 32, further comprising: storing the ambient condition data and the images on the apparatus.

Clause 34. A non-transitory computer-readable storage medium storing one or more programs, the one or more programs comprising instructions that, when executed by an apparatus, cause the apparatus to perform the method of any of clauses 20-33.

Although some of various drawings illustrate a number of logical stages in a particular order, stages that are not order dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages could be implemented in hardware, firmware, software or any combination thereof.

It will also be understood that, although the terms first, second, etc. are, in some instances, used herein to describe various elements, these elements should not be limited by these terms. These terms are only used to distinguish one element from another. For example, a first sensor could be termed a second sensor, and, similarly, a second sensor could be termed a first sensor, without departing from the scope of the various described implementations. The first sensor and the second sensor are both sensors, but they are not the same type of sensor.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations were chosen in order to best explain the principles underlying the claims and their practical applications, to thereby enable others skilled in the art to best use the implementations with various modifications as are suited to the particular uses contemplated.

What is claimed is:

1. An apparatus, comprising:
a support structure;
a plurality of sensors positioned on the support structure;
a camera positioned on the support structure, the camera having a field of view that includes an eye of a user of the apparatus;
one or more processors; and
memory storing instructions that, when executed by the one or more processors, cause the processors to:
detect ambient condition data using the plurality of sensors;
capture imaging data that includes the eye using the camera;
determine a first predefined state of the eye based on the detected ambient condition data and the captured imaging data; and
dispense a fluid proximate to the eye of the user in accordance with the first predefined state of the eye.

2. The apparatus of claim 1, further comprising one or more liquid reservoirs positioned on the support structure;
wherein dispensing the fluid proximate to the eye of the user comprises dispensing the fluid from the one or more liquid reservoirs.

3. The apparatus of claim 2, further comprising one or more agitators positioned in proximity to the one or more liquid reservoirs;
wherein dispensing the fluid proximate to the eye of the user further comprises agitating the fluid in the one or more liquid reservoirs prior to the dispensing the fluid.

4. The apparatus of claim 3, wherein the one or more agitators include one or more of: a radio frequency resonator, a magnetic mixer, and an ultrasonic vibrator.

5. The apparatus of claim 2, wherein:
the one or more liquid reservoirs include one or more micro-heaters; and
the memory further includes instructions that, when executed by the one or more processors, cause the processors to adjust a temperature of the fluid in the one or more liquid reservoirs using the one or more micro-heaters.

6. The apparatus of claim 5, wherein the memory further includes instructions that, when executed by the one or more processors, cause the processors to:
dispense the fluid from the one or more liquid reservoirs by evaporating the fluid using the one or more micro heaters.

7. The apparatus of claim 2, wherein:
the one or more liquid reservoirs comprise a plurality of liquid reservoirs, each of the liquid reservoirs containing a distinct fluid having a corresponding fluid type; and
the memory further includes instructions that, when executed by the one or more processors, cause the processors to:
in accordance with the determined first predefined state of the eye:
identify one or more fluid types corresponding to the first predefined state; and
dispense from the plurality of liquid reservoirs fluids one or more fluids corresponding to the identified fluid types.

8. The apparatus of claim 1, wherein:
the ambient condition data includes two or more of: a light level, an air pressure, humidity, an air flow, and temperature; and
the plurality of sensors include two or more of:
a light sensor for measuring the light level;
an ambient pressure sensor for measuring the air pressure;
a humidity sensor for measuring the humidity;
an airflow sensor for measuring the air flow; and
a temperature sensor for measuring the temperature.

9. The apparatus of claim 1, further comprising:
a tonometer for measuring a pressure of the eye.

10. The apparatus of claim 9, wherein the tonometer comprises:
a first component for deflecting a cornea of the eye; and
a second component for measuring the deflection.

11. The apparatus of claim 1, further comprising a refractor for measuring an intraocular pressure of the eye.

12. The apparatus of claim 1, wherein the memory further includes instructions that, when executed by the one or more processors, cause the processors to:
determine one or more parameters from the imaging data, the one or more parameters including:
a blinking rate of the eye;
a color of the eye;
secretion from the eye;
swelling of the eye;
a size of a pupil of the eye; and
cloudiness of the eye.

13. The apparatus of claim 1, wherein the support structure includes an engagement mechanism for engaging the support structure in a vicinity of the eye.

14. The apparatus of claim 1, further comprising one or more lenses mounted on the support structure.

15. The apparatus of claim 14, wherein:
the one or more lenses include photochromic lenses; and
the memory further includes instructions that, when executed by the one or more processors, cause the processors to vary a shade of the photochromic lenses in accordance with the determined first predefined state of the eye.

16. The apparatus of claim 14, wherein the one or more lenses are configured to display one or more indications to the user, including: light signals, text, and/or images.

17. The apparatus of claim 1, further comprising:
communication circuitry for communicatively connecting the apparatus with an electronic device; and
the memory further includes instructions that, when executed by the one or more processors, cause the processors to:
transmit the ambient condition data and/or the images to the electronic device for display on the electronic device.

18. The apparatus of claim 17, wherein the memory further includes instructions that, when executed by the one or more processors, cause the processors to:
store the ambient condition data and the images on the apparatus.

19. A method, comprising:
at an apparatus having a support structure, a plurality of sensors positioned on the support structure, a camera positioned on the support structure, the camera having a field of view that includes an eye of a user, one or more processors, and memory storing one or more programs configured for execution by the one or more processors, the method comprising:
detecting ambient condition data using the plurality of sensors;
capturing imaging data that includes the eye using the camera;
determining a first predefined state of the eye based on the detected ambient condition data and the captured imaging data; and
dispensing a fluid proximate to the eye in accordance with the first predefined state of the eye.

20. A non-transitory computer-readable storage medium storing instructions that, when executed by one or more processors of an apparatus that comprises a support structure, a plurality of sensors positioned on the support structure, a camera positioned on the support structure, the camera having a field of view that includes an eye of a user, and memory, cause the processors to perform operations comprising:
detecting ambient condition data using the plurality of sensors;
capturing imaging data that includes the eye using the camera;
determining a first predefined state of the eye based on the detected ambient condition data and the captured imaging data; and
dispensing a fluid proximate to the eye in accordance with the first predefined state of the eye.
